(19) United States
(12) Patent Application Publication
(10) Pub. No.: US 2020/0160994 A1
(43) Pub. Date: May 21, 2020

(54) OCCUPANT INJURY DETERMINATION SYSTEMS FOR VEHICLES AND METHODS INCORPORATING THE SAME

(71) Applicant: Toyota Motor North America, Inc., Plano, TX (US)

(72) Inventor: Shintaro Iwaasa, Frisco, TX (US)

(73) Assignee: Toyota Motor North America, Inc., Plano, TX (US)

(21) Appl. No.: 16/193,624

(22) Filed: Nov. 16, 2018

(51) Int. Cl. G16H 50/20

(52) U.S. Cl. CPC G16H 50/20; G16H 10/60

(57) ABSTRACT

A system that improves a determination of estimated injury severity that receives data corresponding to a vehicle at a first server computing device upon occurrence of an event, processes the data through an injury severity algorithm to estimate an occupant injury severity, and transmits the data and the estimated occupant injury severity to a provider computing device and a second sever computing device. The system receives a modified injury severity algorithm at the first server computing device from the second server computing device, the modified injury severity algorithm determined by comparing the estimated occupant injury severity to an actual occupant injury severity that is determined through an assessment of the occupant injury severity by the provider computing device. The modified injury severity algorithm is determined through a comparison of the estimated occupant injury severity to the actual occupant injury severity by the second server computing device.

### TECHNICAL FIELD

The present specification generally relates to systems and methods that sense vehicle characteristics, and more specifically, to crash injury severity determination systems in vehicles and methods for determining the severity of injury of vehicle occupant(s) at the time of a crash.

### BACKGROUND

Occupants of an automotive vehicle may become injured during the event of a crash. In instances of an automobile crash, the injuries that the occupants of the vehicle may sustain can potentially be life-threatening such that receiving medical attention is of notable importance to improve the occupant's likelihood of surviving the injuries. A determination of the severity of an occupant's injuries may not be known until well after the occurrence of the crash, and in some instances not until the occupant has been transported to a local care facility (e.g., hospital). The potential delay in identifying the injuries of an occupant involved in a crash may be detrimental to the occupant's ability to recover.

In some instances, automotive vehicles may include systems programmed to transmit data of a crash to a local care facility for quick assessment of a severity of the injury sustained by an occupant of the vehicle. In response, a local care facility may respond accordingly by preparing the necessary arrangements to aid an occupant based on the injury severity, such as dispatching an ambulance or a helicopter to the crash site. The estimated determination of an occupant's injury based on the transmitted crash data may at times be inaccurate relative to an actual severity of the occupant's injuries, as subsequently identified by medical personnel caring for the occupant at the local care facility. Inaccuracies of estimating an injury severity may be detrimental in providing proper and care for an occupant involved in a crash, and may involve an inappropriate use of resources.

### SUMMARY

In one embodiment, a method of improving a determination of estimated injury severity including receiving data corresponding to a vehicle at a first server computing device of an injury determination system from a local component of the injury determination system upon occurrence of an event in the vehicle. The first server computing device is remotely located from the local component. The method includes processing, by the first server computing device of the injury determination system, the data through an injury severity algorithm to estimate an occupant injury severity, and transmitting, by the first server computing device of the injury determination system, the data and the estimated occupant injury severity to a provider computing device and a second sever computing device of the injury determination system. The provider computing device and the second server computing device are remotely located from the local component. The method further includes receiving, at the first server computing device of the injury determination system, a modified injury severity algorithm from the second server computing device. The modified injury severity algorithm determined by comparing the estimated occupant injury severity to an actual occupant injury severity. The actual occupant injury severity is determined through an assessment of the occupant injury severity by the provider computing device of the injury determination system, and the modified injury severity algorithm is determined through a comparison of the estimated occupant injury severity to the actual occupant injury severity by the second server computing device of the injury sever determination system.

In another embodiment, an injury determination system for estimating an occupant injury severity including a vehicle including a local component and one or more sensors positioned on the vehicle that are communicatively coupled to the local component. The one or more sensors receive data corresponding to sensed properties of the vehicle. The system includes a first server computing device communicatively coupled to the local component of the vehicle such that the local component transmits the data corresponding to the sensed properties of the vehicle to the first server computing device. The first server computing device processes the data through an injury severity algorithm to estimate the occupant injury severity. The system further includes a second server computing device communicatively coupled to the first server computing device such that the first server computing device transmits the data and the estimated occupant injury severity to the second computing device. The second computing device compares the estimated occupant injury severity to an actual occupant injury severity to identify a variance and adjust the injury severity algorithm to thereby minimize the variance.

In another embodiment, a computing device for improving an estimated injury severity algorithm, including a processor and a computer-readable medium including one or more programming instructions that, when executed by the processor, cause the processor to receive non-private data corresponding to a crash event and analyze an estimated injury severity determination to an actual injury severity determination. The estimated injury severity determination is determined by a server computing device and the actual injury determination is determined by a provider computing device, each of which are communicatively coupled to the processor. The processor determines a variance between the estimated injury severity determination and the actual injury severity determination, modifies the estimated injury severity algorithm pursuant to the variance to thereby improve subsequent estimated injury severity determinations, and transmits the modified estimated injury severity algorithm to the server computing device.

These and additional features provided by the embodiments described herein will be more fully understood in view of the following detailed description, in conjunction with the drawings.

### BRIEF DESCRIPTION OF THE DRAWINGS

The embodiments set forth in the drawings are illustrative and exemplary in nature and not intended to limit the subject matter defined by the claims. The following detailed description of the illustrative embodiments can be understood when read in conjunction with the following drawings, where like structure is indicated with like reference numerals and in which:

FIG. 1 schematically depicts an illustrative network of components for an injury determination system according to one or more embodiments shown and described herein;

FIG. 2 schematically depicts illustrative hardware components of a vehicle used in an injury determination system according to one or more embodiments shown and described herein;

FIG. 3 schematically depicts illustrative hardware components of an estimation server computing device used in an injury determination system according to one or more embodiments shown and described herein;

FIG. 4 schematically depicts illustrative hardware components of an optimization server computing device used in an injury determination system according to one or more embodiments shown and described herein; and

FIG. 5 depicts a flow diagram of an illustrative method of improving a determination of estimated injury severity according to one or more embodiments shown and described herein.

### DETAILED DESCRIPTION

The present disclosure relates generally to systems and methods that provide real-time injury severity estimations for occupants involved in automotive crashes. More specifically the present disclosure relates to systems and methods that compare estimated injury severity data to actual injury severity data to improve an accuracy of the estimation formula utilized by the systems and methods when performing subsequent injury severity estimations for future crashes. The injury determination systems are particularly useful for providing a service provider (e.g., a local hospital, a clinic, a healthcare facility, and/or the like) with an indication of the number of individuals involved in a crash and an accurate estimation of a severity of their injuries. Providing a system that allows a service provider to anticipate prospective patients from a nearby crash, and the extent of their injuries, may assist medical personnel in making the necessary preparations for aiding the prospective patient once they arrive to the service provider's location. The injury determination system further provides appropriate emergency response services to an occupant(s) of a vehicle that is involved in a crash. Improving the injury determination system's accuracy in estimating the severity of injuries to the occupants of a crash is vital in efficiently achieving the purposes described above.

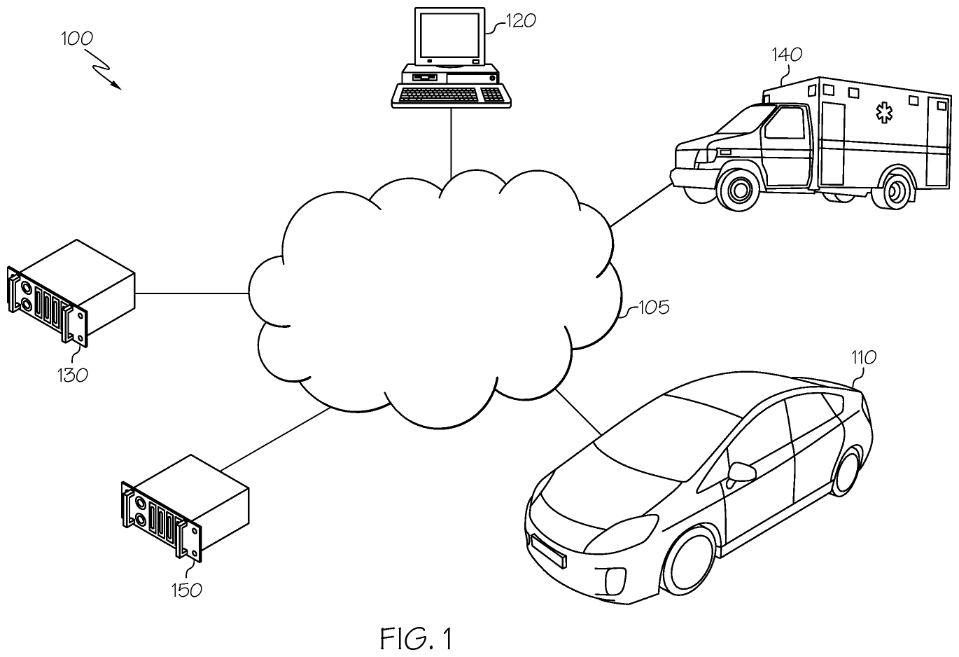

Referring now to the drawings, FIG. 1 depicts an illustrative network having components for an injury determination system 100 according to embodiments shown and described herein. As illustrated in FIG. 1, the injury determination system 100 utilizes a computer network 105 that may include a wide area network (WAN), such as the Internet, a local area network (LAN), a mobile communications network, a public service telephone network (PSTN), a personal area network (PAN), a metropolitan area network (MAN), a virtual private network (VPN), and/or another network. The computer network 105 may generally electronically connect one or more components, systems, and/or devices of the injury determination system 100, such as computing devices and/or components thereof. Illustrative systems, components, and/or devices of the injury determination system 100 may include, but are not limited to, a vehicle 110, a provider computing device 120, an estimation server computing device 130 (e.g., a first server computing device), an emergency response vehicle 140, and an optimization server computing device 150 (e.g., a second server computing device).

The vehicle 110 may generally be any vehicle with one or more onboard computing devices, particularly computing devices that contain hardware for processing data received from sensors positioned on or within the vehicle 110, as described in greater detail herein. In addition, the computing devices may contain hardware for interacting with the other components of the vehicle 100 and/or a user of the vehicle 110, where the devices may be operable to communicate a notification when data is transmitted from the vehicle 110 to a remote server (e.g., estimation server computing device 130). It should be understood that while FIG. 1 only depicts a single vehicle 110 for the purposes of simplicity, the present disclosure is not limited to such. That is, the injury determination system 100 may include a plurality of vehicles 110 such that the injury severity estimation and improvement operabilities of the injury determination system 100 can be simultaneously implemented for each of the plurality of vehicles 110.

It should be understood that the vehicle 110 is merely an illustrative example of a system that contains a local component 200 communicatively coupled to the components, devices, and servers of the injury determination system 100 according to the embodiments described herein. That is, other mobility apparatuses may be monitored in lieu of a vehicle 110 without departing from the scope of the present disclosure. For example, apparatuses such as watercrafts, aircrafts, autonomous vehicle systems, motorcycles, motorized scooters, bicycles, and/or the like may also be a part of the injury determination system described herein. In some embodiments, various local infrastructure systems and/or devices may be communicatively coupled to the injury determination system 100, such as, for example, traffic cameras, roadway sensors, and the like.

The provider computing device 120 is a computing device that provides an interface between a service provider (e.g., a local hospital) and the other components of the injury determination system 100 via the computer network 105. The provider computing device 120 may be used to perform one or more user-facing functions of the injury determination system 100, such as allowing a user to analyze data received from another component of the injury determination system 100 (e.g., an estimation server computing device 130) or inputting information to be transmitted to other components of the injury determination system 100 (e.g., an emergency response vehicle 140, and/or an optimization server computing device 150), as described in greater detail herein. Accordingly, the provider computing device 120 may include at least a display and/or input hardware for facilitating the one or more user-interfacing functions, as described in greater detail herein. The provider computing device 120 may also be used to input additional data into the injury determination system 100 that supplements the data stored and received from the estimation server computing device 130. For example, the provider computing device 120 may contain software programming or the like that allows a user to view crash data detected by a plurality of sensors 250 positioned on each of a plurality of vehicles (including vehicle 110), review an estimated occupant injury severity calculated by the estimation server computing device 130, and provide supplemental information accordingly, such as an actual occupant injury severity, as described in greater detail herein.

The estimation server computing device 130 may receive data from one or more sources (e.g., the vehicle 110 and/or the optimization server computing device 150), analyze the received data, generate data (e.g., an estimated injury severity determination), store data, index data, search data, and/or provide data to the provider computing device 120 and/or the optimization server computing device 150 (or components thereof). More specifically, the estimation server computing device 130 may employ one or more injury severity estimation algorithms for the purposes of analyzing data that is received from the vehicle 110, as described in greater detail herein.

The optimization server computing device 150 may receive data from one or more sources (e.g., the estimation server computing device 130 and/or the provider computing device 120), analyze the received data, generate data (e.g., an improved injury severity algorithm), store data, index data, search data, and/or provide data to the provider computing device 120 and/or the estimation server computing device 130 (or components thereof). More specifically, the optimization server computing device 150 may employ one or more injury severity optimization algorithms for the purposes of improving the injury severity estimation formula utilized by the estimation server computing device 130, as described in greater detail herein.

It should be understood that while the provider computing device 120 is depicted as a personal computer and the server computing devices 130, 150 are depicted as servers, these are nonlimiting examples. In some embodiments, any type of computing device (e.g., mobile computing device, computer, server, cloud-based network of devices, etc.) may be used for any of these components. Additionally, while each of these computing devices is illustrated in FIG. 1 as a single piece of hardware, this is also merely an example. Each of the provider computing device 120 and the server computing devices 130, 150 may represent a plurality of computers, servers, databases, components, and/or the like.

Still referring to FIG. 1, the emergency response vehicle 140 of the injury determination system 100 may generally be any vehicle used to provide assistance to individuals involved in an event, such as a crash. In the present example, the emergency response vehicle 140 is an ambulance that responds to medical emergencies and used for transporting medical patients to treatment (e.g., to a service provider, such as a local hospital). The emergency response vehicle 140 and/or the occupants within the emergency response vehicle may generally have a communications device, such as an onboard radio, a mobile device, and/or the like for receiving information relating to the occurrence of an event involving the vehicle 110. For instance, the emergency response vehicle 140 may be in contact with the provider computing device 120 such that the provider computing device 120 provides a notification of the injury severity and location of occupants in the vehicle 110 when data is transmitted through the injury determination system 100 to the provider computing device 120. It should be understood that while FIG. 1 only depicts a single emergency response vehicle 140 for the purposes of simplicity, the present disclosure is not limited to such. That is, the injury determination system 100 may include a plurality of emergency response vehicles 140 such that the injury severity estimation and improvement operabilities of the injury determination system 100 can be simultaneously implemented for each of the plurality of emergency response vehicles 140.

It should be understood that the emergency response vehicle 140 is merely an illustrative example of a system used to provide assistance to individuals involved in an event and that contains communication means for receiving information from other components, devices, and servers of the injury determination system 100 according to the embodiments described herein. That is, other assistive mobility apparatuses may be incorporated in lieu of or in addition to an emergency response vehicle 140 without departing from the scope of the present disclosure. For example, apparatuses such as a police vehicle, aircraft (e.g., helicopters and/or airplanes), autonomous vehicle systems, watercraft, a motorcycle, a bicycle, and/or the like may also be a part of the injury determination system described herein.

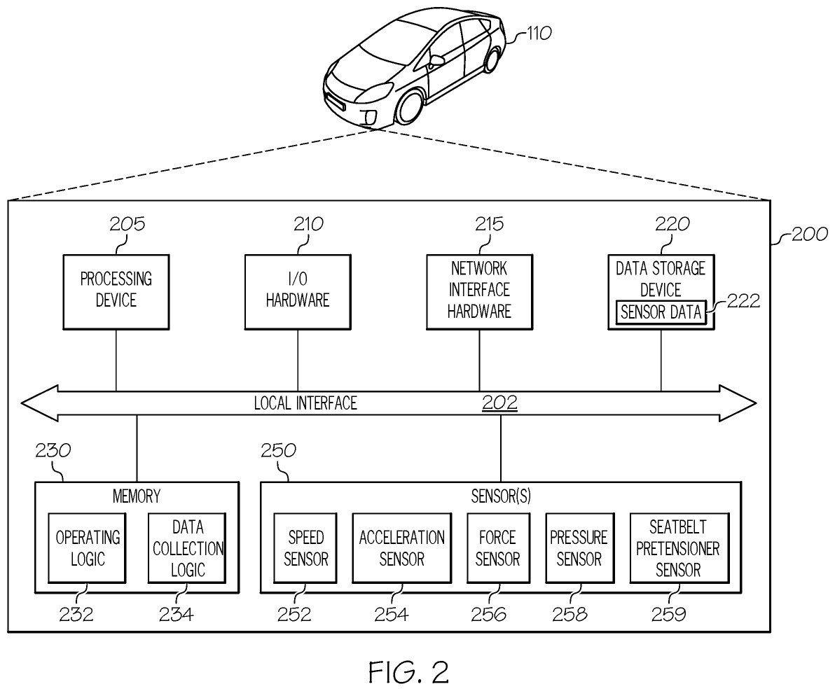

FIG. 2 schematically depicts illustrative hardware components of the vehicle 110 that may be integrated and used in the injury determination system. In particular, the vehicle 110 may include a local component 200 (which may also be referred to as a vehicle component) having a non-transitory computer-readable medium for completing the various processes described herein, embodied as hardware, software, and/or firmware, according to embodiments shown and described herein. The local component 200 may generally be an onboard vehicle computing system. In some embodiments, the local component 200 may be a plurality of vehicle computing systems. While in some embodiments the local component 200 may be configured as a general purpose computer with the requisite hardware, software, and/or firmware, in other embodiments, the local component 200 may also be configured as a special purpose computer designed specifically for performing the functionality described herein. In embodiments where the local component 200 is a general purpose computer, the methods described herein provide a mechanism for improving the functionality of the local component 200 by moving certain processor-intensive tasks away from the local component 200 to be completed by an external device that is more adapted for such tasks (e.g., the server computing devices 130, 150).

The local component 200 may include, for example, a processing device 205, I/O hardware 210, network interface hardware 215, a data storage device 220, a non-transitory memory component 230, and/or one or more sensors 250. A local interface 202, such as a bus or the like, may interconnect the various components. The processing device 205, such as a computer processing unit (CPU), may be the central processing unit of the local component 200, performing calculations and logic operations to execute a program. The processing device 205, alone or in conjunction with the other components, is an illustrative processing device, computing device, processor, or combination thereof. The processing device 205 may include any processing component configured to receive and execute instructions (such as from the data storage device 220 and/or the non-transitory memory component 230).

The non-transitory memory component 230 may be configured as a volatile and/or a nonvolatile computer-readable medium and, as such, may include random access memory (including SRAM, DRAM, and/or other types of random access memory), read only memory (ROM), flash memory, registers, compact discs (CD), digital versatile discs (DVD), and/or other types of storage components. The non-transitory memory component 230 may include one or more programming instructions thereon that, when executed by the processing device 205, cause the processing device 205 to complete various processes, such as certain processes described herein with respect to recording and transmitting crash data to a server computing device upon detecting an occurrence of a crash event. Still referring to FIG. 2, the programming instructions stored on the non-transitory memory component 230 may be embodied as a plurality of software logic modules, where each logic module provides programming instructions for completing one or more tasks.

In some embodiments, the program instructions contained on the non-transitory memory component 230 may be embodied as a plurality of software modules, where each module provides programming instructions for completing one or more tasks. For example, FIG. 2 schematically depicts the non-transitory memory component 230 containing illustrative logic components according to one or more embodiments shown and described herein. As shown in FIG. 2, the non-transitory memory component 230 may be configured to store various processing logic, such as, for example, operating logic 232 and/or data collection logic 234 (each of which may be embodied as a computer program, firmware, or hardware, as an example). The operating logic 232 may include an operating system and/or other software for managing components of the local component 200. The data collection logic 234 may contain one or more software modules for collecting data from one or more sources of the injury determination system (e.g., the one or more sensors 250 and/or the like), converting data, transmitting data, and/or analyzing data, as described in greater detail herein.

The network interface hardware 215 may include any wired or wireless networking hardware, such as a modem, LAN port, wireless fidelity (Wi-Fi) card, WiMax card, mobile communications hardware, and/or other hardware for communicating with other networks and/or devices. For example, the network interface hardware 215 may provide a communications link between the vehicle 110 and the other components of the computer network 105 depicted in FIG. 1, including (but not limited to) the estimation server computing device 130.

Still referring to FIG. 2, the network interface hardware 215 of the local component 200 is operable to transmit crash data including private and non-private information relating to the vehicle 110 in response to the occurrence of a crash involving the vehicle 110, as described in greater detail herein. In this instance, the processing device 205 of the local component 200 executes transmission of the crash data to the estimation server computing device 130 via the network interface hardware 215.

The data storage device 220, which may generally be a storage medium, may contain one or more data repositories for storing data that is received and/or generated. The data storage device 220 may be any physical storage medium, including, but not limited to, a hard disk drive (HDD), memory, removable storage, and/or the like. While the data storage device 220 is depicted as a local device, it should be understood that the data storage device 220 may be a remote storage device, such as, for example, a server computing device or the like (e.g., server computing devices 130, 150).

FIG. 2 further schematically depicts a block diagram of various types of data contained within a storage device (e.g., the data storage device 220) of the local component 200 according to one or more embodiments shown and described herein. The data storage device 220 may include, for example, sensor data 222 that may include, for example, data received from the one or more sensors 250 of the vehicle 110, data manually inputted by a user of the vehicle 110, and/or the like. It should be understood that in other embodiments, the data storage device 220 of the local component 200 may include additional data not depicted and described herein. The I/O hardware 210 may communicate information between the local interface 202 and one or more other components of the vehicle 110. For example, the I/O hardware 210 may act as an interface between the local component 200 and other components, such as mobile phone systems, infotainment systems, and/or the like. In some embodiments, the I/O hardware 210 may be utilized to transmit one or more commands to the other components of the vehicle 110 based on one or more inputs received from a user via other monitoring components, such as, for example, an input from a user of the vehicle 110 confirming that a crash event has occurred.

The one or more sensors 250 may include various hardware components for sensing characteristics of certain vehicle components, particularly sensed properties of the vehicle 110 during the occurrence of an event involving the vehicle 110, such as, for example, a crash. By way of an illustrative example only, the vehicle 110 may include a plurality of sensors 250, such as a speed sensor, an acceleration/deceleration sensor, a force/impact sensor, a pressure sensor(s), a seatbelt pretensioner sensor, and/or a GPS location sensor. In the present example, the plurality of sensors 250 may be programmed and operable to detect, measure, and record data in response to the occurrence of an event involving the vehicle 100 (e.g., a crash). In this instance, the processing device 205 of the local component 200 is operable to initiate activation of the plurality of sensors 250 during identification of a crash occurrence involving the vehicle 110.

In the present example, a speed sensor 252 is operable to measure and record a speed of the vehicle 110 immediately before and/or during the occurrence of a crash. An acceleration sensor 254 is operable to measure and record a rate of acceleration of the vehicle 110 immediately before and/or during the occurrence of a crash. Additionally, the acceleration sensor 254 is further operable to measure and record a rate of deceleration of the vehicle 110 immediately after and/or during the occurrence of the crash. A force/impact sensor 256 is operable to measure and record an amount of force (i.e., a force acting on a body as a result of acceleration or gravity) impacted upon a body of the vehicle 110 at the time of a crash. Further, a pressure sensor 258 is operable to identify a weight of an occupant seated within the vehicle 110 at the occurrence of the crash. It should be understood that the vehicle 110 may include multiple pressure sensors 258, and in particular, include a pressure sensor 258 at each respective seat within the vehicle 110. In this instance, each pressure sensor 258 is operable to detect and identify whether an occupant is seated within the vehicle 110, at the respective seat that the pressure sensor 258 is located at, when the vehicle 110 is involved in a crash. Additionally, the pressure sensor 258 may be operable to measure a weight of the occupant seated within the respective seat of the vehicle 110 at which the pressure sensor 258 is located. In this instance, the weight of the occupant as measured by the pressure sensor 258 may be processed and correlated to an estimated age and/or gender of the occupant seated within the vehicle 110 at the respective seat (e.g., such as by processing device 205, and/or the estimation server computing device 130).

As merely an illustrative example, the data collection logic 234 of the vehicle 110 may include programming instructions that, when executed by the processing device 205, correlate a measured weight of about 55 kilograms (i.e., 120 pounds) or less to a fully grown female occupant and/or a teenage child occupant and a measured weight of about 77 kilograms (i.e., 170 pounds.) or more to a fully grown male occupant. For example, the pressure sensor 258 may detect a measured weight of about 50 kilograms. at a particular seat within the vehicle 110 such that the data collection logic 234 correlates an identification of the occupant seated thereon as that of an adult female or teenage child. The data collection logic 234 may further analyze other data detected by the plurality of sensors 250 and/or other components of the vehicle 110 to further determine the identification of the occupant. For instance, data collected by an image sensor positioned within the cabin of the vehicle 110, data processed by a voice recognition software communicatively coupled to a microphone positioned within the cabin of the vehicle 110, data received by a user input at the local component 200 of the vehicle 110, and/or the like may be further analyzed by the data collection logic 234 in conjunction with the measured weight data to accurately determine an identification (i.e., an age, a gender, and the like) of the occupant.

By determining an identification of the occupant, the estimation server computing device 130 may accurately estimate an injury severity of the occupant relative to the crash characteristics of the crash event upon receiving the data from the vehicle 110. For instance, the estimation server computing device 130 may determine that an injury severity of an adult female occupant based on the measured force applied to the vehicle 110, as detected by the force sensor 256, is less severe than an injury severity to a teenage child occupant involved in an identical crash and experiencing a similar amount of force due to the variance between the physical bodily development of the adult occupant relative to the teenage child occupant. In other words, data relating to an age and/or gender of an occupant is indicative of the occupant's bodily development and ability to safely absorb the energy generated from a crash event (i.e., an impact force). Accordingly, in the context of the other sensed characteristics relating to the vehicle 110 and the crash event (e.g., speed, acceleration, force impact, and the like), identifying an age and/or gender of an occupant may influence an estimation of the occupant's injury severity. It should be understood that the data collection logic 234 and/or the estimation server computing device 130 may be programmed with various other weight correlation parameters than that described above.

Additionally, a seatbelt pretensioner sensor 259 is operable to sense whether the seatbelt pretensioner for a respective seatbelt within the vehicle 110 was activated during the occurrence of the crash. In this instance, the seatbelt pretensioner sensor 259 is operable to detect and identify whether an occupant is seated within the vehicle 110, at the respective seat at which the seatbelt pretensioner is located, when the vehicle 110 is involved in a crash and the seatbelt pretensioner sensor 259 of the particular seat becomes activated. In other embodiments, the seatbelt pretensioner sensor 259 may be operable to detect a force in which a seatbelt is pulled during the occurrence of a crash. In this instance, the strength at which an occupant is held to securely maintain the occupant within a seat of the vehicle 110 may be indicative of an impact/force applied to the vehicle 110 during the crash.

It should be understood that the plurality of sensors 250 are otherwise not limited by the present disclosure such that the vehicle 110 may include additional or fewer sensors 250 therein. For example, the plurality of sensors 250 may further include electrical sensors, temperature sensors, and the like. Illustrative characteristics that may be sensed by the plurality of sensors 250 include, but are not limited to, current, voltage, resistance, temperature, and the like. Each of the plurality of sensors 250 may be positioned on, integrated with, positioned in line with, or positioned adjacent to one or more features or devices of the vehicle 110 that are to be measured. One or more sensors 250 may generally be configured to sense one or more characteristics or properties of the vehicle 110 during a crash, and transmit the crash data corresponding to the one or more sensed characteristics to another component of the injury determination system to be used to estimate an occupant injury severity, as described in greater detail herein. For example, the one or more characteristics or properties sensed by the one or more sensors 250 of the vehicle 110 may include sensing whether an airbag of the vehicle 110 has been deployed, whether a seat belt of the vehicle 110 was buckled during the crash event, and the like.

It should further be understood that the components of the local component 200 of the vehicle 110 illustrated in FIG. 2 are merely illustrative and are not intended to limit the scope of this disclosure. More specifically, while the components in FIG. 2 are illustrated as residing within the local component 200 of the vehicle 110, this is a nonlimiting example. In some embodiments, one or more of the components may reside external to the local component 200 and/or the vehicle 110.

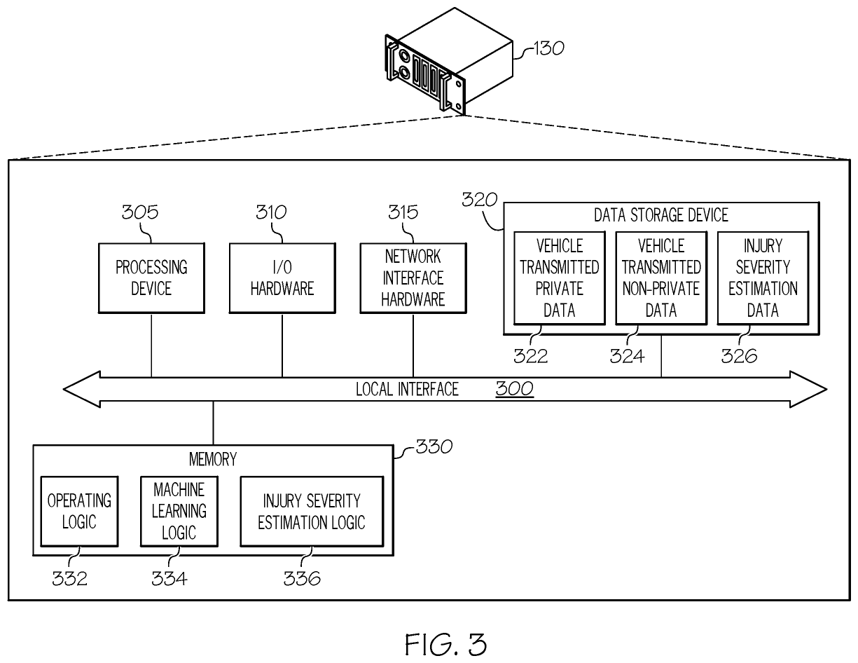

FIG. 3 schematically depicts illustrative hardware components of the estimation server computing device 130 that may be used in receiving crash data from the local component 200 of the vehicle 110, analyzing the crash data, and estimating an injury severity for each of the occupants seated within the vehicle 110 by executing an injury severity algorithm as described in greater detail herein. It should be understood that in the present example the estimation server computing device 130 is remotely located relative to the local component 200 of the vehicle 110.

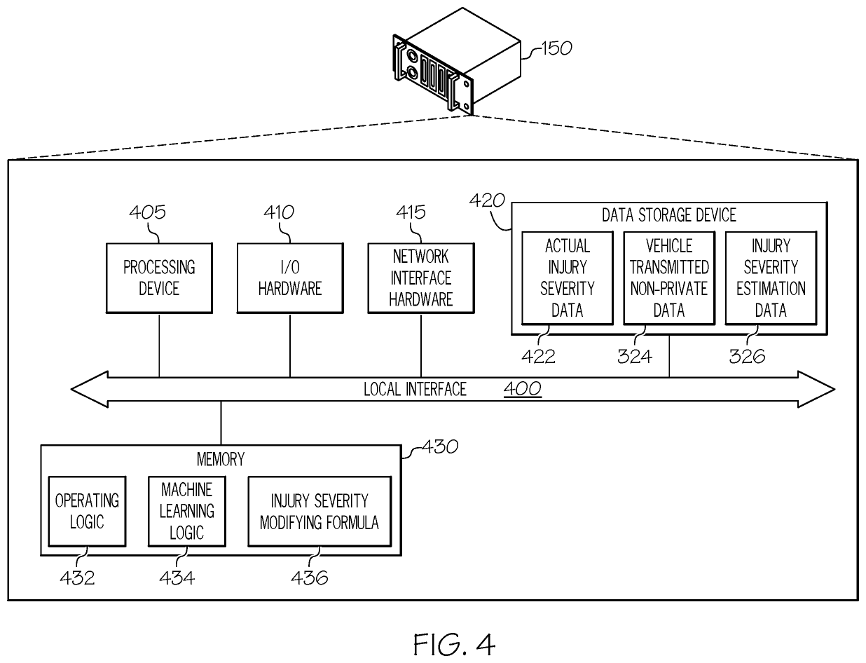

The estimation server computing device 130 may include a non-transitory computer-readable medium for completing the various processes described herein, embodied as hardware, software, and/or firmware, according to embodiments shown and described herein. While in some embodiments the estimation server computing device 130 may be configured as a general purpose computer with the requisite hardware, software, and/or firmware, in other embodiments, the estimation server computing device 130 may also be configured as a special purpose computer designed specifically for performing the functionality described herein. In embodiments where the estimation server computing device 130 is a general purpose computer, the methods described herein provide a mechanism for improving the functionality of the estimation server computing device 130 by moving certain less processor-intensive tasks away from the estimation server computing device 130 to be completed by an external device or server (e.g., the second server computing device of FIG. 4) that may be more adapted for such tasks so as to speed up the process of estimating injury severities of occupants in a vehicle 110 that are involved in a crash, and to avoid having the estimation server computing device 130 complete unnecessary computing tasks.

Still referring to FIG. 3, the estimation server computing device 130 may include a processing device 305, I/O hardware 310, network interface hardware 315, a data storage device 320, and a non-transitory memory component 330. A local interface 300, such as a bus or the like, may interconnect the various components. The processing device 305, such as a computer processing unit (CPU), may be the central processing unit of the estimation server computing device 130, performing calculations and logic operations to execute a program. The processing device 305, alone or in conjunction with the other components, is an illustrative processing device, computing device, processor, or combination thereof. The processing device 305 may include any processing component configured to receive and execute instructions (such as from the data storage device 320 and/or the non-transitory memory component 330).

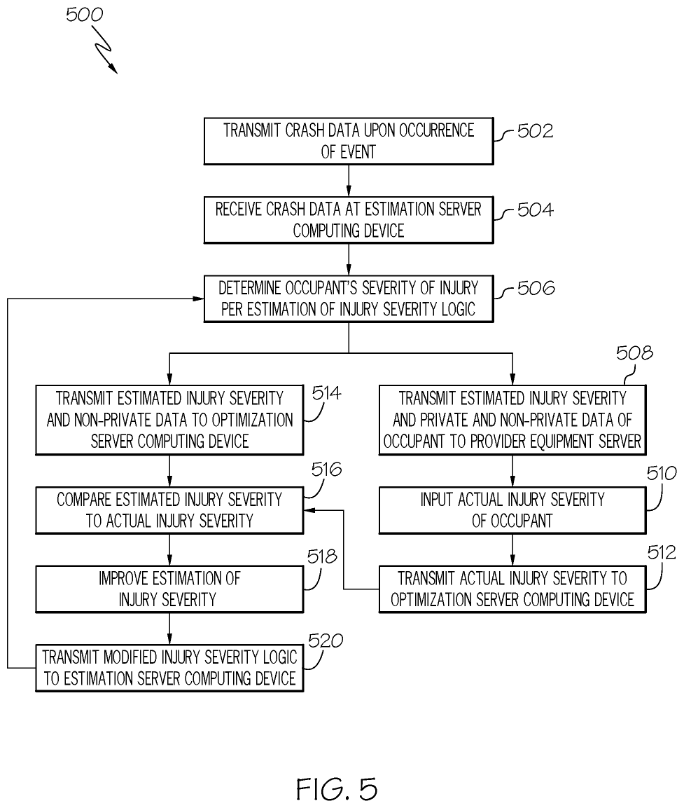

The memory component 330 may be configured as a volatile and/or a nonvolatile computer-readable medium and, as such, may include random access memory (including SRAM, DRAM, and/or other types of random access memory), read only memory (ROM), flash memory, registers, compact discs (CD), digital versatile discs (DVD), and/or other types of storage components. The memory component 330 may include one or more programming instructions thereon that, when executed by the processing device 305, cause the processing device 305 to complete various processes, such as, for example, certain processes described herein with respect to FIG. 5. The programming instructions stored on the memory component 330 may be embodied as a plurality of software logic modules, where each logic module provides programming instructions for completing one or more tasks, as described in greater detail below.

The network interface hardware 315 may include any wired or wireless networking hardware, such as a modem, LAN port, wireless fidelity (Wi-Fi) card, WiMax card, mobile communications hardware, and/or other hardware for communicating with other networks and/or devices. For example, the network interface hardware 315 may provide a communications link between the estimation server computing device 130 and the other components of the computer network 105 depicted in FIG. 1, including (but not limited to) the vehicle 110, the provider computing device 120, the optimization server computing device 150, and/or the like.

Still referring to FIG. 3, the data storage device 320, which may generally be a storage medium, may contain one or more data repositories for storing data that is received and/or generated. The data storage device 320 may be any physical storage medium, including, but not limited to, a hard disk drive (HDD), memory, removable storage, and/or the like. While the data storage device 320 is depicted as a local device, it should be understood that the data storage device 320 may be a remote storage device, such as, for example, on another server computing device or the like. Illustrative data that may be contained within the data storage device 320.

The data storage device 320 may include, for example, vehicle-transmitted private data 322, vehicle-transmitted non-private data 324, and/or injury severity estimation data 326. Vehicle-transmitted private data 322 may include, for example, data received from the vehicle 110 (FIG. 1) and/or a component thereof (e.g., the local component 200) relating to private, identifiable information relating to the vehicle 110 and/or the occupant(s) seated therein during the occurrence of a crash. It should be understood that at least a portion of the vehicle-transmitted private data 322 may be identified by at least one or more of the sensors 250 (FIG. 2) located on the vehicle 110. By way of merely an illustrative example, such vehicle-transmitted private data 322 may include a vehicle identification number (VIN) of the vehicle 110 involved in the crash; a GPS location of the vehicle 110 at the time of the crash event (as detected by a GPS location sensor included in the vehicle 110); an age, gender and/or weight of the occupant(s) seated within the vehicle 110 at the time of the crash (as detected by the pressure sensor(s) 258 included in the vehicle 110), identifying information pertaining to an owner and/or occupant of the vehicle 110, and/or the like. It should be understood that additional or fewer vehicle-transmitted private data 322 may be transmitted from the vehicle 110 to the estimation server computing device 130 than those described herein.

The vehicle-transmitted non-private data 324 may include, for example, data received from the vehicle 110 (FIG. 1) and/or a component thereof (e.g., the local component 200) relating to non-private, unidentifiable information relating to the vehicle 110 and/or the occupant(s) seated therein during the occurrence of a crash. It should be understood that at least a portion of the vehicle-transmitted non-private data 324 may be identified by at least one or more of the sensors 250 located on the vehicle 110. By way of merely an illustrative example, such vehicle-transmitted non-private data 324 may include a speed of the vehicle 110 during the occurrence of the crash (as detected by the speed sensor 252 located on the vehicle 110), an acceleration/deceleration of the vehicle 110 before and/or after the occurrence of the crash (as detected by the acceleration sensor 254 located on the vehicle 110), an amount of force impacted and/or endured by a body of the vehicle 110 during the occurrence of the crash (as detected by the force/impact sensor 256 located on the vehicle 110), a number of occupants seated within the vehicle 110 at the time of the crash (as detected by the pressure sensor(s) 258 included in the vehicle 110), an activation of a seatbelt pretensioner(s) within the vehicle 110 during the occurrence of the crash (as detected by the seatbelt pretensioner sensor(s) 259 located on the vehicle 110). It should be understood that additional or fewer vehicle-transmitted non-private data 324 may be transmitted from the vehicle 110 to the estimation server computing device 130 than those described herein.

It should be further understood that separating private data from non-private data relating to a crash event is necessary to perform an optimization of an injury severity algorithm utilized by an accident report service system, such as the injury determination system 100. In particular, to protect the privacy (including medical information) of individuals involved in a crash event, an accident report service system may not access private data of the victims involved in the crash when generating accident reports. Accordingly, an analysis and improvement of an injury severity algorithm used by the injury determination system 100, to estimate injury severities of the occupants involved in the crash, is not possible at either the estimation server computing device 130 or the provider computing device 120 since both of these devices 120, 130 of the injury determination system 100 receive private data relating to the crash event. Accordingly, as described in greater detail herein, an optimization of the injury severity algorithm (as employed by the estimation server computing device 130) requires a comparison of the estimated injury severity data and the actual injury severity data at a third device that does not receive privacy data of the occupants involved in the crash (e.g., the optimization server computing device 150).

The injury severity estimation data 326 may be data that is generated as a result of the estimation server computing device 130 analyzing the vehicle-transmitted private data 322 and the vehicle-transmitted non-private data 324 received from the vehicle 110. In particular, the injury severity estimation data 326 is data that is generated as a result of an estimation of an injury severity for each of the occupants included in the vehicle 110 during the occurrence of the crash. The injury severity estimation data 326 is generated based on the processing device 305 executing the injury severity estimation logic 336 while factoring in the vehicle-transmitted data 322, 324 received from the vehicle 110. As will be described in greater detail herein, upon generating the injury severity estimation data 326, the estimation server computing device 130 is operable to transmit the injury severity estimation data 326 for each occupant involved in the crash of the vehicle 110 to at least the provider computing device 120 and the optimization server computing device 150.

Still referring to FIG. 3, the I/O hardware 310 may communicate information between the local interface 300 and one or more other components of the estimation server computing device 130. In some embodiments, the I/O hardware 310 may be utilized to transmit one or more commands to the other components of the estimation server computing device 130 based on data received from the local component 200 of the vehicle 110 and/or the optimization server computing device 150.

In some embodiments, the program instructions contained on the memory component 330 may be embodied as a plurality of software modules, where each module provides programming instructions for completing one or more tasks. For example, FIG. 3 schematically depicts the memory component 330 containing illustrative logic components according to one or more embodiments shown and described herein. The memory component 330 may be configured to store various processing logic, such as, for example, operating logic 332, machine learning logic 334, and/or injury severity estimation logic 336 (each of which may be embodied as a computer program, firmware, or hardware, as an example). The operating logic 332 may include an operating system and/or other software for managing components of the estimation server computing device 130.

The machine learning logic 334 may contain one or more software modules for obtaining data from the vehicle 110, analyzing the data (i.e., crash data), and determining information from the data to provide to the provider computing device 120 and/or the optimization server computing device 150, as described in greater detail herein. By way of example, the machine learning logic 334 includes software modules for analyzing the crash data recorded by the one or more sensors 250 and determining various information from the analysis using a machine learning algorithm now known or later developed, such as, for example, linear regression, logistic regression, linear discriminant analysis, classification and regression trees, naïve bayes, K-Nearest Neighbors (KNN), learning vector quantization, support vector machines, bagging and random forest, boosting and Adaboost, and the like. Illustrative information that may be determined by the machine learning logic 334 includes, but is not limited to, a total number of occupants within the vehicle 110 at the time of the crash, a respective age and/or gender of the occupants, a classification of the crash data as either private or non-private information, and the like. Alternatively, it should be understood that in other embodiments the analysis of the crash data may be performed by one or more software modules of the data collection logic 234 of the vehicle 110.

The injury severity estimation logic 336 (also referred to as an injury severity algorithm and/or formula) may contain one or more software modules for processing the data that is received from the vehicle 110 (and/or the local component 200) and transmitting the processed data (e.g., in the form of private information, non-private information, and an estimated injury severity determination) to the provider computing device 120 and the optimization server computing device 150 for additional processing, as described in greater detail herein. For example, one or more software modules of the injury severity estimation logic 336 may include mathematical formulas that correlate an impact force sustained by an occupant's body relative to the physical bodily development of the occupant to thereby estimate a severity of injury to the occupant. As merely an illustrative example, the formulas implemented in the injury severity estimation logic 336 may include parameters such as, but not limited to, a mass (e.g., a weight) of the occupant (as measured by the pressure sensor 258), a speed of the vehicle 110 at the time of the crash (as measured by the speed sensor 252), and an elapsed stopping distance of the vehicle 110 after the occurrence of the crash event (as determined from measuring a deceleration of the vehicle 110 by the acceleration/deceleration sensor 254). In the present example, the injury severity estimation logic 336 applies a formula to determine an impact force “F” endured by a body of the occupant, such as, for example, F=−(MV²/2D), where “M” corresponds to the occupant's mass, “V” corresponds to the vehicle's 110 traveling speed, and “D” corresponds to the elapsed stopping distance of the vehicle 110. In this instance, the injury severity estimation logic 336 determines a severity of injury to the occupant based on the identification of the crash characteristics (i.e., force endured by the body of the occupant) and the personal characteristics of the occupant (i.e., the age and/or gender of the occupant enduring the force).

It should be understood that the injury severity estimation logic 336 may implement various other formulas than those described and shown herein to determine an impact force sustained by an occupant and/or to estimate an injury severity of an occupant. It should further be understood that the components illustrated in FIG. 3 are merely illustrative and are not intended to limit the scope of this disclosure. More specifically, while the components in FIG. 3 are illustrated as residing within the estimation server computing device 130, this is a nonlimiting example. In some embodiments, one or more of the components may reside external to the estimation server computing device 130.

FIG. 4 schematically depicts illustrative hardware components of the optimization server computing device 150 that may be used in receiving crash data from the estimation server computing device 130 and the provider computing device 120, receiving the estimated injury severity determination(s) from the estimation server computing device 130 (i.e., the injury severity estimation data 326 for each occupant within the vehicle 110), receiving the actual injury severity determination(s) from the provider computing device 120 (i.e., the actual injury severity data 422), analyzing the collective data, and identifying a variance (i.e., inaccuracy) between the estimated injury severity and the actual injury severity for each of the occupants seated within the vehicle 110 by executing an injury severity modification algorithm as described in greater detail herein. It should be understood that in the present example the optimization server computing device 150 is remotely located relative to the local component 200 of the vehicle 110 and the estimation server computing device 130. It should be further understood that the optimization server computing device 150 of the present example is substantially similar to the estimation server computing device 130 described above except for the differences explicitly noted herein.

In particular, the optimization server computing device 150 includes a local interface 400, a processing device 405, a I/O hardware 410, a network interface hardware 415, a data storage device 420, and a memory component 430 that are configured and operable substantially similar to those same components of the estimation server computing device 130. However, the data storage device 420 of the optimization server computing device 150 includes, for example, in addition to the vehicle-transmitted non-private data 324 and the injury severity estimation data 326 received from the estimation server computing device 130, actual injury severity data 422 received from the provider computing device 120, as described in greater detail herein. It should be understood that the data storage device 420 of the optimization server computing device 150 differs from the data storage device 320 of the estimation server computing device 130 in that the optimization server computing device 150 does not include vehicle transmitted private data. Accordingly, unlike the estimation server computing device 130, the optimization server computing device 150 does not receive the vehicle-transmitted private data 322 transmitted from the vehicle 110.

Still referring to FIG. 4, the memory component 430 of the optimization server computing device 150 may be configured to store various processing logic, such as, for example, operating logic 432, machine learning logic 434, and/or injury severity modifying formula 436 (each of which may be embodied as a computer program, firmware, or hardware, as an example). The operating logic 432 may include an operating system and/or other software for managing components of the optimization server computing device 150. The machine learning logic 434 may contain one or more software modules for obtaining data relating to the vehicle 110 and/or the occupants seated therein at the time of the crash event, analyzing the collective data, and determining information from the data to provide to the estimation server computing device 130, as described in greater detail herein. In particular, dissimilar to the machine learning logic 334 of the estimation server computing device 130 (FIG. 3), the machine learning logic 434 of the optimization server computing device 150 only obtains vehicle-transmitted non-private data 324 to the exclusion of vehicle-transmitted private data 322 relating to the vehicle 110 and/or the occupants seated therein. In addition, the machine learning logic 434 obtains the injury severity estimation data 326 generated by the estimation server computing device 130 and the actual injury severity data 422 generated by the provider computing device 120.

By way of example, the machine learning logic 434 includes one or more software modules for analyzing the non-private crash data generated by the devices 120, 130 (e.g., vehicle-transmitted non-private data 324, estimated injury severity data 326, actual injury severity data 422, and the like) to determine various information from said analysis. For example, the machine learning logic 434 of the optimization server computing device 150 is operable to determine a discrepancy between the estimated injury severity data 326 generated by the estimation server computing device 130, based at least partly on the vehicle-transmitted non-private data 324, and the actual injury severity data 522 generated by the provider computing device 120. The injury severity modifying formula 436 (also referred to as a modified injury severity algorithm) may contain one or more software modules for processing the discrepancy between the estimated injury severity data 326 and the actual injury severity data 522 that is received from the estimation server computing device 130 and the provider computing device 120, respectively, and modifying the injury severity estimation logic 336 utilized by the estimation server computing device 130 to minimize said discrepancy. The operating logic 432 of the optimization server computing device 150 may further include software for transmitting the processed data (e.g., in the form of a modified injury severity algorithm) to the estimation server computing device 130 for future use, as described in greater detail herein.

As mentioned above, the various components of the injury determination system 100 described with respect to FIGS. 2-4 may be used to carry out one or more processes and/or provide functionality for receiving crash data from the one or more sensors 250 of the vehicle 110, processing the crash data, and determining an estimated injury severity for each of the occupants within the vehicle 110 from the processed crash data such that an appropriate response action can be undertaken by the provider computing device 120 and/or the emergency response vehicle 140. The various components of the injury determination system 100 may further be used to carry out one or more processes and/or provide functionality for improving an accuracy of the estimated injury severity determination, and in particular, the injury severity formula utilized by the estimation server computing device 130 by modifying the estimated injury severity algorithm. An illustrative example of the various processes is described with respect to FIG. 5.

Referring also to FIGS. 1-4, FIG. 5 schematically depicts a flow diagram of an illustrative method 500 of modifying an injury severity algorithm by a plurality of devices to thereby improve the estimated injury severity determination by the injury determination system 100. Initially, a vehicle 110 is involved in an event, such as, in the present example, a crash such that the one or more sensors 250 located on the vehicle 110 record data relating to the crash event. In particular, the processing device 205 of the local component 200 executes the data collection logic 234 stored within the non-transitory memory component 230 to store the data detected by the sensors 250 of the vehicle 110 in response to detecting the occurrence of the crash event. Accordingly, the one or more sensors 250 detect and record one or more data points pertaining to the vehicle 110, such as a speed, acceleration, deceleration, impact, airbag deployment, seatbelt engagement, and the like as described in greater detail above. The one or more sensors 250 further detect and record one or more data points pertaining the occupant(s) seated within the vehicle 110 at the time of the crash, such as a number, age, gender, weight, and the like of the occupant(s) as described in greater detail above.

The sensor data 222 recorded by the one or more sensors 250) relating to the crash is thereby stored in the data storage device 220 of the local component 200 and includes both private data and non-private data. At block 502, the vehicle 110, and in particular, the processing device 205 of the local component 200 executes the operating logic 232 to transmit crash data to the estimation server computing device 130. It should be understood that the crash data includes the sensor data 222 and other data relating to the vehicle 110 and/or the crash event. For example, the crash data may include a Vehicle Identification Number (VIN) of the vehicle 110, a license plate number of the vehicle 110, an identification of the registered owner(s) of the vehicle 110, and/or other private and/or non-private data relating to the vehicle 110 and/or the crash event that is not detected by the sensors 250. The processing device 205 transmits the sensor data 222 through the computer network 105 via the network interface hardware 215 of the vehicle 110.

At block 504, the crash data is received at the estimation server computing device 130, and in particular, the estimation server computing device 130 communicates with the vehicle 110 and receives the data through the network interface hardware 315 of the estimation server computing device 130. In this instance, the processing device 305 of the estimation server computing device 130 executes machine learning logic 334 to collect, analyze, and categorize the data transmitted by the vehicle 110. In particular, the machine learning logic 334 includes instructions that cause the processing device 305 to analyze, categorize, and store the data in the data storage device 320 as vehicle-transmitted private data 322 and vehicle-transmitted non-private data 324. The vehicle-transmitted private data 322 may include, for example, a VIN of the vehicle 110, an age, gender or weight of the occupant(s) seated within the vehicle 110, the GPS location of the vehicle 110 at the time of the crash and/or the like. The vehicle-transmitted non-private data 324 may include, for example, a speed of the vehicle 110 at the time of the crash, a force impact endured by the vehicle 110 during the crash, a number of occupants seated within the vehicle 110 and/or the like.

The machine learning logic 334 further analyzes the crash data to translate the vehicle-transmitted data 322, 324 into various characteristics relating to the vehicle 110, the occupant(s) seated therein, and/or the crash event. For instance, the machine learning logic 334 analyzes the vehicle-transmitted data 322, 324 to identify personal characteristics relating to the occupant(s) of the vehicle 110 and crash characteristics relating to the vehicle 110 for use by the injury severity estimation logic 336 to estimate an injury severity of each of the occupants within the vehicle 110. Personal characteristics of an occupant may include an occupant's age, gender, weight, location relative to the cabin of the vehicle 110, and the like, as determined by the vehicle-transmitted data 322, 324 detected by the one or more pressure sensors 258. As described in detail above, one or more pressure sensors 258 may be positioned at one or more seats within the vehicle 110 such that a location of an occupant seated thereon is determined upon detecting the presence of a pressure by the pressure sensor 258. Further, the pressure sensor 258 measures a weight of the occupant seated within the respective seat of the vehicle 110 at which the pressure sensor 258 is located. In this instance, the weight of the occupant is processed and correlated by the data collection logic 234 to an estimated age and/or gender of the occupant seated within the vehicle 110 based on programmed parameters of the data collection logic 234, as described herein.

Crash characteristics of the vehicle 110 may include a make or model of the vehicle 110, which may be indicative of a size, shape, and/or strength of pillar reinforcement structures of the vehicle 110. Crash characteristics may further include a speed of the vehicle 110 (as measured by the speed sensor 252), a degree of impact/force applied to the vehicle 110 and a direction and/or location of impact/force applied to the vehicle 110 relative to a position of the occupant(s) in the cabin of the vehicle 110 (as measured by the force sensor 256), a deployment of airbags within the vehicle 110 (as measured by an airbag sensor), use of seatbelts by the occupant(s) in the vehicle 110 (as measured by the seatbelt pretensioner sensor 259), and the like during the crash event, as detected by the one or more sensors 250.

At block 506, the injury severity estimation logic 336 of the estimation server computing device 130 causes the processing device 305 to process the crash data (i.e., vehicle-transmitted data 322, 324) analyzed by the machine learning logic 334 to thereby estimate an injury severity for each occupant seated within the vehicle 110 during the occurrence of the crash. In particular, the injury severity estimation logic 336 evaluates the personal characteristics of the occupant and the crash characteristics of the vehicle 110 when determining an injury severity estimation. Accordingly, the injury severity estimation logic 336 is operable to estimate a severity of injury to a particular occupant within the vehicle 110 based on the known characteristics of the occupant, the vehicle 110, and the crash event. As merely an illustrative example, a vehicle 110 traveling at about 96 kilometers per hour (i.e., about 60 miles per hour), that decelerates at a rate such that the elapsed stopping distance is about 0.3 meters (i.e., 1 foot) will dictate varying injury severities upon an occupant seated within the vehicle 110 based on an age, gender, and/or position of the occupant seated within the vehicle 110. In a first instance, with an occupant having a weight of about 54.5 kilograms (i.e., 120 pounds) and enduring an impact force of about 7.2 tons (i.e., 14,451 pounds), the injury severity estimation logic 336 may estimate life-threatening injuries to the occupant based on the personal characteristics of the occupant. In contrast, in a second instance, with an occupant having a weight of about 72.6 kilograms (i.e., 160 pounds) and enduring an impact force of about 9.6 tons (i.e., 19,268 pounds), the injury severity estimation logic 336 may estimate moderately severe injuries to the occupant relative to the occupant weighing about 54.5 kilograms despite involving similar crash characteristics relating to the vehicle 110 and the crash event.

Upon estimating the injury severity determination for the occupant(s) of the vehicle 110, the operating logic 332 of the estimation server computing device 130 causes the processing device 305 to store the injury severity estimation data 326 in the data storage device 320. With the injury severity estimation data 326 computed by the estimation server computing device 130, the processing device 305 executes the operating logic 332 to transmit the vehicle-transmitted private data 322, the vehicle-transmitted non-private data 324, and/or the injury severity estimation data 326 to the other components of the injury determination system 100. In this instance, the estimation server computing device 130 simultaneously transmits the estimated injury severity, the private data, and/or the non-private data to the provider computing device 120 at block 508 and the optimization server computing device 150 at block 514 for processing and subsequent analysis as described in greater detail herein.

At block 508, the processing device 305 transmits the vehicle-transmitted private data 322, the vehicle-transmitted non-private data 324, and the injury severity estimation data 326 to the provider computing device 120 through the computer network 105, via the network interface hardware 315 of the estimation server computing device 130. It should be understood that the provider computing device 120 may be a computing device positioned and located at a local service provider responsible for providing services in response to an occurrence of the event. In the present example, the service provider is a local hospital that provides medical services to patients in response to events, such as crashes involving vehicles (like vehicle 110). Upon receiving the data at the provider computing device 120 from the estimation server computing device 130, the medical personnel at the service provider may be informed of the injury severities of the individuals involved in the event (i.e., the occupants of the vehicle 110 involved in the crash). By providing this information soon after the occurrence of the crash, the medical personnel of the service provider may perform the necessary preparations for aiding the patients once they arrive to the location of the service provider. In some examples, the injury determination system 100, and in particular the estimation server computing device 130, provides a specific bodily injury designation to the provider computing device 120, such as, for example, a fractured bone, a lacerated skin region, a punctured organ, and/or the like. It should be understood that the bodily injury designations described above are merely for illustrative purposes and various other injury designators and/or information may be generated by the estimation server computing device 130.

Additionally, the provider computing device 120 of the service provider may communicate with the emergency response vehicle 140 via the computer network 105 to thereby facilitate a response measure that corresponds with the estimated occupant injury severity involved in the crash. The injury determination system 100 thus provides appropriate emergency response services to an occupant(s) of the vehicle 110 involved in the crash by the provider computing device 120 communicating with the emergency response vehicle 140 based on the estimated injury severity determination computed by the estimation server computing device 130.

For example, in some instances the provider computing device 120 may classify the estimated occupant injury severity to be life-threatening such that the provider computing device 120 directs an emergency response vehicle 140 in the form of an aircraft (e.g., helicopter) to be sent to the crash site due to the severe nature of the injuries involved. In this instance, an aircraft may be a more appropriate response measure than other emergency response vehicles, such as an ambulance, due to the relatively quicker transportation means provided by an aircraft which, when an occupant of a crash event has sustained life-threatening injuries, is capable of traveling to and from the crash site with relative ease and quicker efficiency. This may be desirable to ensure the occupant receives medical attention prior to the severity of the occupant's injuries increasing.

In other instances, the provider computing device 120 may classify the estimated occupant injury severity to be non-life threatening such that the provider computing device 120 directs an emergency response vehicle 140 in the form of an ambulance to be sent to the crash site due to the non-severe nature of the injuries involved. It should be understood that in some instances the provider computing device 120 may classify the estimated occupant injury severity, as computed by the estimation server computing device 130, to be minor such that the emergency response vehicle 140 is not required to be directed toward the crash site.

At block 510, the provider computing device 120 determines the actual injury severity of the occupant(s) involved in the crash by receiving an input and/or signal that is indicative of the actual injury severity of the occupant(s), thereby generating the actual injury severity data 422 at the provider computing device 120. At block 512, the actual injury severity data 422 of the occupant(s) is transmitted from the provider computing device 120 of the injury determination system 100 to the optimization server computing device 150 via the computer network 105.

As discussed above, the estimation server computing device 130 simultaneously transmits the estimated injury severity data 326, the vehicle-transmitted private data 322, and/or the vehicle-transmitted non-private data 324 to the provider computing device 120 and the optimization server computing device 150 for processing and subsequent analysis. At block 514, with the injury severity estimation data 326 computed by the estimation server computing device 130, the processing device 305 of the estimation server computing device 130 executes the operating logic 332 to transmit the vehicle-transmitted non-private data 324 and the injury severity estimation data 326 to the optimization server computing device 150 through the computer network 105 via the network interface hardware 315 of the estimation server computing device 130. Accordingly, the optimization server computing device 150 receives the vehicle-transmitted non-private data 324 and the injury severity estimation data 326 from the estimation server computing device 130, and the actual injury severity data 422 from the provider computing device 120.

It should be understood that the optimization server computing device 150 does not receive the vehicle-transmitted private data 322 from either the estimation server computing device 130 and/or the provider computing device 120. In this instance, the optimization server computing device 150 solely analyzes the vehicle-transmitted non-private data 324, the injury severity estimation data 326, and the actual injury severity data 422 when performing the following analysis described herein. As described in detail above, by not receiving the vehicle-transmitted private data 322 at the optimization server computing device 150, the optimization server computing device 150 is capable of analyzing and modifying the injury severity estimation logic 336 employed by the estimation server computing device 130. In particular, it should be understood that accident report service systems are not permitted to access privacy data of individuals involved in a crash, such as those included in vehicle-transmitted private data 322. Accordingly, the vehicle-transmitted private data 322 is excluded from being transmitted to the optimization server computing device 150 to thereby allow for the analysis and improvement of the algorithm used by the injury determination system 100 (i.e., the injury severity estimation logic 336) to preform accident report services. In some embodiments, the processing device 305 of the estimation server computing device 130 executes the operating logic 332 to redact the vehicle-transmitted private data 322 prior to transmitting the crash data to the optimization server computing device 150.

At block 516, the machine learning logic 534 of the optimization server computing device 150 causes the processing device 505 to analyze and compare the estimated injury severity data 326 to the actual injury severity data 422. In particular, the processing device 505 executes the machine learning logic 534 to measure and identify a quantifiable variance between the estimated injury severity data 326 and the actual injury severity data 422 relative to the vehicle-transmitted non-private data 324 that the estimated injury severity data 326 was based on. For example, the machine learning logic 434 initially identifies a discrepancy between the actual injury severity of an occupant (i.e., the actual injury severity data 422) and the estimated injury severity of the occupant (i.e., the injury severity estimation data 326) that is quantified to a measureable variance. Subsequently, the machine learning logic 434 analyzes the vehicle-transmitted non-private data 324, which was initially evaluated by the estimation server computing device 130 to generate the estimated injury severity data 326, to identify an existing correlation between the current formula of the injury severity estimation logic 336 and the vehicle-transmitted non-private data 324 that attributed to generating the injury severity estimation. In other words, the machine learning logic 434 includes one or more software modules that, when executed by the processing device 405, causes the optimization server computing device 150 to examine the parameters that form the injury severity estimation logic 336 to determine points of deviations and/or inaccuracies in the formula of the injury severity estimation logic 336. It should be understood that the machine learning logic 434 of the optimization server computing device 150 identifies the points deviations and/or inaccuracies in the formula of the injury severity estimation logic 336 of the estimation server computing device 130 to optimize and improve an accuracy of the injury severity estimation logic 336 on subsequent injury severity estimations performed by the estimation server computing device 130.

At block 518, upon calculating the variance between the estimated injury severity data 326 and the actual injury severity data 522 and identifying formulistic imperfections in the injury severity estimation logic 336 based on the calculated variance, the processing device 405 of the optimization server computing device 150 executes the injury severity modifying formula 436 to construct a modified injury severity algorithm that minimizes the variance between the estimated injury severity data 326 and the actual injury severity data 422. In particular, the injury severity modifying formula 436 adjusts the injury severity estimation logic 336 based on difference between the estimated injury severity data 326 relative to the actual injury severity data 422 to thereby improve an accuracy of the injury severity estimation logic 336 utilized by the estimation server computing device 130. For example, one or more formula parameters of the injury severity estimation logic 336 are selectively modified by the processing device 405 of the optimization server computing device 150 such that an updated injury severity estimation, based on identical vehicle-transmitted non-private data 324, generates an improved (i.e., more accurate) estimation of an occupant's injury severity relative to the actual injury severity data 422, thereby minimizing a variance between the estimated injury severity and the actual injury severity.

At block 520, the processing device 405 of the optimization server computing device 150 transmits the improved, modified injury severity algorithm to the estimation server computing device 130. In this instance, the original injury severity estimation algorithm is replaced by the modified injury severity algorithm such that subsequent injury severity estimations performed by the estimation server computing device 130 utilizes the modified injury severity algorithm. It should be understood that the illustrative method 500 of the injury determination system 100 may be routinely performed during continued occurrences of an event (e.g., crash) involving vehicles 110 communicatively coupled to the injury determination system 100 such that the injury severity estimation algorithm may be continuously modified and improved. Accordingly, the accuracy of the injury determination system 100 in estimating occupant injury severity may be routinely improved through incorporation of the optimization server computing device 150 configured to update the injury severity estimation formula utilized by the estimation server computing device 130.

The above-described system includes components that provide real-time injury severity estimations for occupants involved in crashes when in a vehicle, and more specifically, to systems that compare estimated injury severity data to actual injury severity data to modify the original injury severity estimation formula and thereby improve an accuracy of the estimation formula. The injury determination system includes server computing devices that are remotely coupled to the vehicle involved in the crash and a provider computing device of a local service provider. The system facilitates transmission of information to a service provider of the severity of injury for the occupants involved in the crash to thereby assist medical personnel of the service provider in preparing for admission of the necessary medical services. The injury determination system further provides appropriate emergency response services to the occupant(s) of the vehicle involved in the crash.

It is noted that the terms “substantially” and “about” may be utilized herein to represent the inherent degree of uncertainty that may be attributed to any quantitative comparison, value, measurement, or other representation. These terms are also utilized herein to represent the degree by which a quantitative representation may vary from a stated reference without resulting in a change in the basic function of the subject matter at issue.

While particular embodiments have been illustrated and described herein, it should be understood that various other changes and modifications may be made without departing from the spirit and scope of the claimed subject matter. Moreover, although various aspects of the claimed subject matter have been described herein, such aspects need not be utilized in combination. It is therefore intended that the appended claims cover all such changes and modifications that are within the scope of the claimed subject matter.

What is claimed is:

1. A method of improving a determination of estimated injury severity, the method comprising:
receiving data corresponding to a vehicle at a first server computing device of an injury determination system from a local component of the injury determination system upon occurrence of an event with the vehicle, wherein the first server computing device is remotely located from the local component;
processing, by the first server computing device of the injury determination system, the data through an injury severity algorithm to estimate an occupant injury severity;
transmitting, by the first server computing device of the injury determination system, the data and the estimated occupant injury severity to a provider computing device and a second sever computing device of the injury determination system, wherein the provider computing device and the second server computing device are remotely located from the local component; and
receiving, at the first server computing device of the injury determination system, a modified injury severity algorithm from the second server computing device, the modified injury severity algorithm determined by comparing the estimated occupant injury severity to an actual occupant injury severity;
wherein the actual occupant injury severity is determined at the provider computing device of the injury determination system, and the modified injury severity algorithm is determined through a comparison of the estimated occupant injury severity to the actual occupant injury severity by the second server computing device of the injury sever determination system.

2. The method of claim 1, wherein the event is a crash involving the vehicle and the data comprises crash data that includes private information and non-private information relating to the vehicle at a time of the crash.

3. The method of claim 2, further comprising:
analyzing, at the first sever computing device of the injury determination system, the non-private information of the crash data measured by at least one sensor positioned on the vehicle.

4. The method of claim 3, wherein analyzing the non-private information measured by the at least one sensor positioned on the vehicle comprises:
evaluating, at the first server computing device of the injury determination system, a speed of the vehicle during the event;
evaluating, at the first server computing device of the injury determination system, a force impact endured by a body of the vehicle during the event;
evaluating, at the first server computing device of the injury determination system, a weight of each seat within the vehicle during the event to determine a number of occupants within the vehicle;
evaluating, at the first server computing device of the injury determination system, a deployment of an airbag of the vehicle during the event;
evaluating, at the first server computing device of the injury determination system, an activation of a seatbelt pretensioner of the vehicle during the event; and
evaluating, at the first server computing device of the injury determination system, a GPS location of the vehicle during the event.

5. The method of claim 2, wherein transmitting, by the first server computing device of the injury determination system, the data to the provider computing device comprises:
transmitting the private information and the non-private information.

6. The method of claim 2, wherein transmitting, by the first server computing device of the injury determination system, the data to the second server computing device comprises:
transmitting only the non-private information such that the first server computing device redacts the private information from the data transmitted to the second server computing device.

7. The method of claim 1, wherein the comparison of the estimated occupant injury severity to the actual occupant injury severity comprises:
identifying, at the second server computing device of the injury determination system, a variance of the estimated occupant injury severity from the actual occupant injury severity.

8. The method of claim 7, wherein the modified injury severity algorithm received at the first server computing device of the injury determination system is the injury severity algorithm modified by the variance to thereby update subsequent estimations of the occupant injury severity performed by the first server computing device.

9. The method of claim 1, further comprising:
updating, at the first server computing device of the injury determination system, the injury severity algorithm with the modified injury severity algorithm such that the injury severity algorithm is replaced with the modified injury severity algorithm.

10. The method of claim 1, further comprising:
receiving data corresponding to a subsequent vehicle at the first server computing device of the injury determination system from a local component of the subsequent vehicle upon occurrence of a subsequent event in the subsequent vehicle; and
processing, by the first server computing device of the injury determination system, the data through the modified injury severity algorithm to estimate an occupant injury severity.

11. An injury determination system for estimating an occupant injury severity, the system comprising:
a vehicle including a local component and one or more sensors positioned on the vehicle that are communicatively coupled to the local component, wherein the one or more sensors receive data corresponding to sensed properties of the vehicle;
a first server computing device communicatively coupled to the local component of the vehicle such that the local component transmits the data corresponding to the sensed properties of the vehicle to the first server computing device, wherein the first server computing device processes the data through an injury severity algorithm to estimate the occupant injury severity; and
a second server computing device communicatively coupled to the first server computing device such that the first server computing device transmits the data and the estimated occupant injury severity to the second computing device;
wherein the second computing device compares the estimated occupant injury severity to an actual occupant injury severity to identify a variance and adjust the injury severity algorithm employed by the first server computing device to thereby minimize the variance.

12. The injury determination system of claim 11, further comprising a provider computing device communicatively coupled to the first server computing device such that the first server computing device transmits the data and the estimated occupant injury severity to the provider computing device.

13. The injury determination system of claim 12, wherein the provider computing device determines the actual occupant injury severity.

14. The injury determination system of claim 12, wherein the data includes private information and non-private information, and the first server computing device transmits the private information and non-private information to the provider computing device.

15. The injury determination system of claim 14, wherein the first server computing device transmits only the non-private information to the second sever computing device.

16. A computing device for improving an estimated injury severity algorithm, the device comprising:
a processor; and
a computer-readable medium including one or more programming instructions that, when executed by the processor, cause the processor to:
receive non-private data corresponding to a crash event;
analyze an estimated injury severity determination to an actual injury severity determination, wherein the estimated injury severity determination is determined by a server computing device and the actual injury determination is determined by a provider computing device, each of which are communicatively coupled to the processor;
determine a variance between the estimated injury severity determination and the actual injury severity determination; and
modify the estimated injury severity algorithm pursuant to the variance to thereby improve subsequent estimated injury severity determinations; and
transmit the modified estimated injury severity algorithm to the server computing device.

17. The computing device of claim 16, wherein the non-private data corresponding to the crash event are detected by a plurality of sensors positioned along a vehicle involved in the crash event.

18. The computing device of claim 17, wherein the non-private data detected by the plurality of sensors comprises:
a speed of the vehicle during the event;
a force impact endured by a body of the vehicle during the event;
a weight of each seat within the vehicle during the event, to determine a number of occupants within the vehicle;
an activation of an airbag of the vehicle during the event;
an activation of a seatbelt pretensioner of the vehicle during the event; and
a GPS location of the vehicle during the event.

19. The computing device of claim 16, wherein the computing device does not receive private data corresponding to the crash event from the server computing device or the provider computing device.

20. The computing device of claim 16, wherein the provider computing device receives private data and non-private data corresponding to the crash event from the server computing device.
